(19) United States
(12) Patent Application Publication
(10) Pub. No.: US 2005/0169872 A1
(43) Pub. Date: Aug. 4, 2005

(54) Cosmetic compositions, process for its preparation

(75) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/030,193

(22) Filed: Jan. 7, 2005

Related U.S. Application Data
(60) Provisional application No. 60/545,174, filed on Feb. 18, 2004.

(30) Foreign Application Priority Data
Jan. 29, 2004 (FR) 0400849

(51) Int. Cl. A61K 7/06; A61K 7/11

(52) U.S. Cl. 424/70.16

(57) ABSTRACT

The invention relates to a process for preparing a cosmetic composition for treating keratin material by percolating a fluid at a pressure of at least 3 bar through at least one cationic agent for conditioning keratin materials, in solid or pasty form.

### REFERENCE TO PRIOR APPLICATIONS

This application claims priority to U.S. provisional application 60/545,174 filed Feb. 18, 2004, and to French patent application 0400849 filed Jan. 29, 2004, both incorporated herein by reference.

### FIELD OF THE INVENTION

The present invention relates to a composition and a process for preparing it, the composition being useful for the cosmetic treatment of keratin materials, such as the skin and keratin fibres, for example the hair, and also to a cosmetic process for treating keratin materials using this composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials, for example sensitized hair, i.e. hair that has been damaged or embrittled by the chemical action of atmospheric agents and/or hair treatments such as permanent-waving, dyeing or bleaching. Cosmetic treatment compositions comprising conditioning agents that are sparingly soluble or insoluble in water, such as cationic polymers or cationic surfactants, intended to repair or limit the harmful or adverse effects produced by the various treatments or attacking factors to which keratin materials are more less repeatedly subjected, may thus be applied to these materials. These conditioning agents may also improve the cosmetic properties of natural hair.

Conditioning agents such as cationic polymers and cationic surfactants facilitate the disentangling of the hair and make it soft and supple.

These conditioning agents also give the skin cosmetic properties, such as anti-irritant properties.

However, cosmetic treatment compositions containing such conditioning agents are generally aqueous compositions in which the said agents must be dissolved. The lack of solubility of these compounds reduces the conditioning power of these compositions. In addition, this solubility criterion reduces the number of water-insoluble cationic conditioning agents that can be used for the cosmetic treatment of keratin materials. This is particularly the case for compounds with a high melting point.

### DETAILED DESCRIPTION OF THE PREFERRED EMBODIMENTS

The inventor has discovered, surprisingly, that by using a novel process for preparing a cosmetic composition for treating keratin materials, compositions that are more or less concentrated in conditioning agent(s) may be obtained in a very short time, for example of less than 2 minutes, according to need, especially without preserving agent, enabling the solubility problems outlined above to be overcome.

This process may be performed simply. A pressurized fluid, the temperature of which is preferably greater than or equal to 30° C., preferably ranging from 30° C. to 150° C. and even more preferably ranging from 40° C. to 120° C., is passed for a very short time, for example of less than one minute, through at least one cationic conditioning agent in solid or pasty form, preferably in solid form and more preferably in pulverulent form.

It also enables the use in anhydrous form of conditioning agents that are unstable in aqueous compositions either because they react with water or because they react in aqueous solution with compounds that do not react with them in an anhydrous composition.

The compositions prepared according to this process may have limited stability on storage, which is not a drawback in this case since the process leads to a ready-to-use composition intended to be used quickly after its preparation, for example within five minutes of preparation, especially after cooling to a cosmetically acceptable temperature, preferably below 60° C. The composition may be used up to one week after its preparation, or longer, depending on the rate of degradation of the conditioning agent.

Given the very short preparation time, the cosmetic treatment compositions may be prepared “on demand” by mixing the active compounds, i.e. according to the desired cosmetic properties, in a particular embodiment.

According to another embodiment, the cationic conditioning agents may be packaged in a ready-to-use device, and it is not necessary to determine beforehand the concentrations of said agents in solution, which limits the measuring errors by the user.

In addition, the process according to the invention makes it possible to avoid the use of multi-compartment bottles, which makes the process particularly economical and safer for the user.

The composition thus obtained may be used alone or as a mixture with another composition.

A further advantage of this preparation process is the production of compositions that give good cosmetic properties. In particular, keratin fibres treated with a composition obtained via the process according to the invention have good conditioning properties, especially in terms of disentangling, smoothness, sheen and soft feel.

One subject of the invention is thus a process for preparing a cosmetic composition for treating keratin materials, comprising a step of percolating a fluid at a pressure of at least 3 bar through at least one cationic conditioning agent for keratin materials, in solid or pasty form.

Another subject of the invention is a composition that may be obtained via the process according to the invention.

A subject of the invention is also the use of the composition obtained according to the process of the invention for the cosmetic treatment of keratin materials, and especially for conditioning the hair.

Finally, a subject of the invention is a packaging device for performing the preparation process of the present invention.

Other subjects, characteristics, aspects and advantages of the invention will emerge even more clearly on reading the description and the examples that follow.

According to the invention, the invention process comprises percolating a fluid, preferably at a temperature of greater than or equal to 30° C., preferably ranging from 30° C. to 150° C. and better still ranging from 40° C. to 120° C., at a pressure of at least 3 bar (3×10 Pa), through at least one cationic conditioning agent for keratin materials, in solid or pasty form.

Percolation is a movement of fluid through a saturated porous medium, allowing the passage of the fluid due to the action or effect of pressure.

The fluid may be steam optionally accompanied by liquid water, or of one or more cosmetically acceptable, especially organic, liquid and/or gaseous solvents, or alternatively of a mixture of steam optionally accompanied by liquid water, and of one or more cosmetically acceptable liquid and/or gaseous solvents. Preferably, the fluid comprises at least steam possibly accompanied by liquid water, and even more preferably it is steam possibly accompanied by liquid water.

Useful organic solvents that may be mentioned include C₁-C₄ lower alcohols, such as ethanol and isopropanol; polyols and polyol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, and mixtures thereof.

The term “water-insoluble compound” means any compound which, at a concentration of greater than or equal to 0.1% by weight in water at 25° C., does not form an isotropic solution that is transparent to the naked eye.

The cationic conditioning agent is in solid form or in pasty form, preferably in solid form and even more preferably in pulverulent form.

For the purposes of the present invention, the term “pasty form” means a consistency intermediate between a solid phase and a liquid phase. The viscosity of this pasty phase is preferably greater than 0.1 Pa·s and even more preferably greater than 1 Pa·s, at 25° C. with a shear rate of 10 s⁻¹.

The term “keratin materials” means the skin, the scalp, mucous membranes such as the lips, and/or the integuments such as the nails, the eyelashes, the eyebrows and the hair.

The process of the present invention may be performed using a standard device for generating a pressurized fluid, at a temperature preferably of greater than or equal to 30° C. and even more preferably ranging from 30° C. to 150° C. Such a device comprises a pressure-resistant chamber, equipped with a thermal block, and also a circuit for conveying the fluid produced to the cationic conditioning agent.

According to another embodiment, the device comprises a reservoir of liquid(s) and also a pump for conveying the liquid(s) to the chamber.

The liquid contained in the reservoir is either water, or a cosmetically acceptable solvent or a mixture of several cosmetically acceptable solvents, or alternatively a mixture of water and of one or more cosmetically acceptable solvents. In a manner that is particularly preferred in the invention, the liquid is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., preferably ranging from 30° C. to 150° C., under a pressure of at least 4 bar, preferably greater than 10 bar and most particularly between 10 and 30 bar.

The cationic conditioning agent(s), in solid or pasty form, may be used directly in the device for generating the pressurized fluid in a container intended for this use. They may also be packaged in a particular device for packaging a cosmetic composition, comprising a closed housing delimited by at least one wall that is at least partially permeable to a fluid under a pressure of at least 3 bar, the composition containing at least one cationic conditioning agent, in solid or pasty form. Such devices are described, for example, in patent applications WO 00/56629, EP 512 470, WO 99/03753 or U.S. Pat. No. 5,897,899.

According to one particular embodiment, the housing is delimited by two sealed sheets. According to another embodiment, the housing is delimited by a tray closed with a lid.

These devices may be manufactured from woven or nonwoven plastic or plant materials, for example cellulose, metal such as aluminium, or composite materials. Such devices are described, for example, in patent applications WO 00/56629, EP 512 470 or WO 99/03753.

The cationic conditioning agents may be chosen for example from cationic polymers, cationic surfactants, cationic silicones and cationic proteins.

The cationic polymers that may be used in accordance with the present invention include all those already known per se as improving the cosmetic properties of the hair, i.e. especially those described in patent application EP-A-0 337 354 and in French patent applications FR-A-2 270 846, 2 383 660, 2 598 611, 2 470 596 and 2 519 863.

Even more generally, for the purposes of the present invention, the term “cationic polymer” denotes any polymer comprising cationic groups and/or groups that may be ionized into cationic groups.

The cationic polymers that are preferred are chosen from those containing units comprising primary, secondary, tertiary and/or quaternary amine groups that either may form part of the main polymer chain or may be borne by a side substituent directly attached thereto.

The cationic polymers used generally have a number-average or weight-average molar mass ranging from 500 to 5×10⁶ approximately and preferably between 103 and 3×10⁶ approximately.

Among the cationic polymers that may be mentioned more particularly are polymers of the polyamine, polyamino amide and polyquaternary ammonium type. These are known products.

The polymers of the polyamine, polyamido amide and polyquaternary ammonium type that may be used in accordance with the present invention, and that may especially be mentioned, are those described in French patents Nos 2 505 348 and 2 542 997. Among these polymers, mention may be made in particular of:
(1) homopolymers or copolymers derived from acrylic or methacrylic esters or amides and comprising at least one of the units of the following formulae: 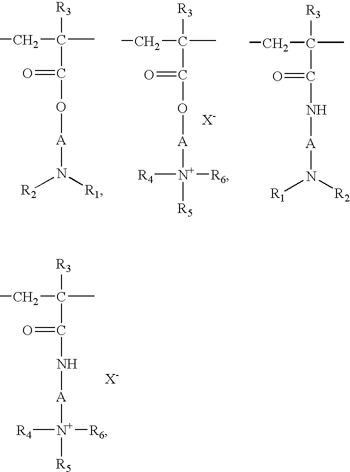
in which:
R₃, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical and preferably an alkyl group containing from 1 to 6 carbon atoms;
R₁ and R₂, which may be identical or different, represent hydrogen or an alkyl group containing from 1 to 6 carbon atoms, and preferably methyl or ethyl;
X⁻ denotes an anion derived from a mineral or organic acid, such as a methosulfate anion or a halide such as chloride or bromide.

The copolymers of family (1) can also contain one or more units derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may preferably be made of:
copolymers of acrylamide and of dimethylaminoethyl methacrylate quaternized with dimethyl sulfate or with a dimethyl halide,
the copolymers of acrylamide and of methacryloyloxy-ethyltrimethylammonium chloride described, for example, in patent application EP-A-080 976,
the copolymer of acrylamide and of methacryloyloxy-ethyltrimethylammonium methosulfate,
quaternized or non-quaternized vinylpyrrolidone/dialkylaminoalkyl acrylate or methacrylate copolymers. These polymers are described in detail in French patents 2 077 143 and 2 393 573,
dimethylaminoethyl methacrylate/vinylcaprolactam/vinylpyrrolidone terpolymers,
vinylpyrrolidone/methacrylamidopropyldimethylamine copolymers,
and quaternized vinylpyrrolidone/dimethylaminopropyl-methacrylamide copolymers.
(2) cationic polysaccharides, especially cationic celluloses and cationic galactomannan gums. Among the cationic polysaccharides that may be mentioned more particularly are cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups, which are described in French patent 1 492 597 and in particular the polymers sold under the names “JR” (JR 400, JR 125, JR 30M) or “LR” (LR 400, LR 30M) by the company Amerchol. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described especially in U.S. Pat. No. 4,131,576 such as hydroxyalkylcelluloses, for instance hydroxymethyl-, hydroxyethyl- or hydroxypropylcelluloses grafted especially with a methacryloyl-ethyltrimethylammonium, methacrylamidopropyltrimethylammonium or dimethyldiallylammonium salt.

Cationic galactomannan gums are described more particularly in U.S. Pat. No. 3,589,578 and U.S. Pat. No. 4,031,307, in particular guar gums containing trialkylammonium cationic groups. Use is made, for example, of guar gums modified with a salt (e.g. chloride) of 2,3-epoxypropyltrimethylammonium.

Such products are sold especially under the names Jaguar C13 S, Jaguar C 15, Jaguar C 17 or Jaguar C162 by the company Rhodia.
(3) polymers of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361;
(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508;
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkyl-enetriamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylaminohydroxypropyl/diethyl-enetriamine polymers.
(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid being between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. No. 3,227,615 and 2,961,347.
(7) cyclopolymers of alkyldiallylamine or of dialkyl-diallylammonium, such as the homopolymers or copolymers containing, as main constituent of the chain, units corresponding to formula (II) or (III): 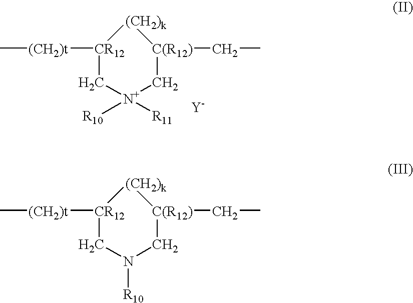
in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer (and its homologues of low weight-average molar mass) and copolymers of diallyldimethylammonium chloride and of acrylamide.
(8) quaternary diammonium polymers containing repeating units corresponding to the formula: 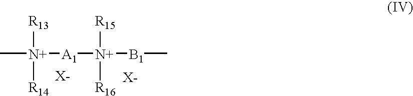
in which formula (IV):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals such as hydroxyethyl, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ—;
in which D denotes:
a) a glycol residue of formula: —O-Z-O—, where Z denotes a linear or branched hydrocarbon-based radical or a group corresponding to one of the following formulae:
   —(CH₂—CH₂-O)ₙ—CH₂—CH₂—
   —[CH₂—CH(CH₃)—O]_{y}—CH₂—CH(CH₃)—
where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical
   —CH₂—CH₂—S—S—CH₂—CH₂—;
d) a ureylene group of formula: —NH—CO—NH—;
Preferably, X⁻ is an anion such as chloride or bromide.

These polymers generally have a number-average molar mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula: 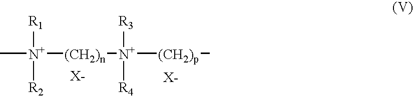
in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

One compound of formula (V) which is particularly preferred is the one for which R₁, R₂, R₃ and R₄ represent a methyl radical and n=3, p=6 and X=Cl, which is known as Hexadimethrine chloride according to the INCI (CTFA) nomenclature.

(9) polyquaternary ammonium polymers consisting of units of formula (VI): 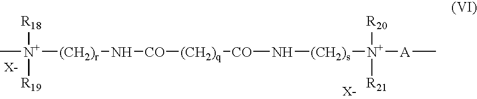
in which formula:
R₁₈, R₁₉, R₂₀ and R₂₁, which may be identical or different, represent a hydrogen atom or a methyl, ethyl, propyl, β-hydroxyethyl, P-hydroxypropyl or —CH₂CH₂(OCH₂CH₂)ₚOH radical,
where p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion such as a halide,
A denotes a dihalide radical or preferably represents —CH₂—CH₂-O—CH₂—CH₂—.

Such compounds are described in particular in patent application EP-A-122 324.
(10) quaternary polymers of vinylpyrrolidone and of vinylimidazole.
(11) polyamines.
(12) preferably crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers that can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes, cationic polyurethanes and chitin derivatives.

Among the cationic polymers mentioned above, which are suitable in the invention, the ones that may preferably be used are quaternary cellulose ether derivatives such as the products sold under the name “JR 400” by the company Amerchol, cationic cyclopolymers, in particular dimethyldiallylammonium chloride homopolymers or copolymers, quaternary polymers of vinylpyrrolidone and of vinylimidazole, optionally crosslinked homopolymers or copolymers of methacryloyloxy(C₁-C₄)alkyltri(C₁-C₄)alkylammonium salts, and mixtures thereof.

Among the cationic surfactants that may be mentioned are quaternary ammonium salts, and examples that may especially be mentioned include:
those of general formula (V) below: 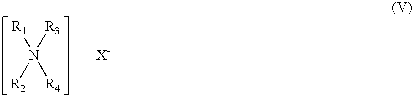
in which the symbols R₁ to R₄, which may be identical or different, represent a linear or branched aliphatic radical containing from 1 to 30 carbon atoms, or an aromatic radical such as aryl or alkylaryl. The aliphatic radicals may comprise hetero atoms such as, especially, oxygen, nitrogen, sulfur and halogens. The aliphatic radicals are chosen, for example, from alkyl, alkoxy, (C₂-C₆) polyoxyalkylene, alkylamide, (C₁₂-C₂₂)alkylamido(C₂-C₆)alkyl, (C₁₂-C₂₂)alkylacetate and hydroxyalkyl radicals, containing from about 1 to 30 carbon atoms; X⁻ is an anion chosen from the group of halides, phosphates, acetates, lactates, (C₂-C₆) alkyl sulfates and alkyl- or alkylaryl-sulfonates;
quaternary ammonium salts of imidazoline, for instance those of formula (VI) below: 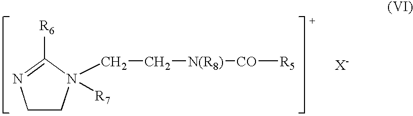
in which R₅ represents an alkenyl or alkyl radical containing from 8 to 30 carbon atoms, for example fatty acid derivatives of tallow or of coconut, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulfates, alkyl sulfonates or alkylaryl sulfonates. R₅ and R₆ preferably denote a mixture of alkenyl or alkyl radicals containing from 12 to 21 carbon atoms, such as, for example, fatty acid derivatives of tallow, R₇ denotes methyl and R₈ denotes hydrogen. Such a product is, for example, Quaternium-27 (CTFA 1997) or Quaternium-83 (CTFA 1997), which are sold under the names “Rewoquat®” W75, W90, W75PG and W75HPG by the company Witco,
diquaternary ammonium salts of formula (VII): 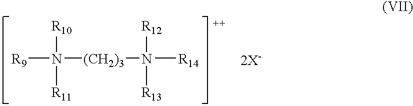
in which R₉ denotes an aliphatic radical containing from about 16 to 30 carbon atoms, R₁₀, R₁₁, R₁₂, R₁₃ and R₁₄, which may be identical or different, are chosen from hydrogen and an alkyl radical containing from 1 to 4 carbon atoms, and X⁻ is an anion chosen from the group of halides, acetates, phosphates, nitrates, ethyl sulfates and methyl sulfates. Such diquaternary ammonium salts in particular comprise propanetallowdiammonium dichloride;
quaternary ammonium salts containing at least one ester function, such as those of formula (VIII) below: 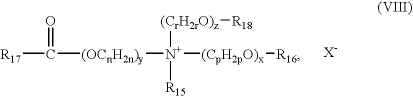
in which:
R₁₅ is chosen from C₁-C₆ alkyl radicals and C₁-C₆ hydroxyalkyl or dihydroxyalkyl radicals;
R₁₆ is chosen from:
a radical 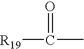
linear or branched, saturated or unsaturated C₁-C₂₂ hydrocarbon-based radicals R₂₀,
a hydrogen atom,
R₁₇ is chosen from:
a radical 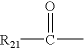
linear or branched, saturated or unsaturated C₁-C₆ hydrocarbon-based radicals R₂₂,
a hydrogen atom,
R₁₇, R₁₉ and R₂₁, which may be identical or different, are chosen from linear or branched, saturated or unsaturated C₇-C₂₁ hydrocarbon-based radicals;
r, n and p, which may be identical or different, are integers ranging from 2 to 6;
y is an integer ranging from 1 to 10;
x and z, which may be identical or different, are integers ranging from 0 to 10;
X⁻ is a simple or complex organic or mineral anion;
with the proviso that the sum x+y+z is from 1 to 15, that when x is 0, then R₁₆ denotes R₂₀ and that when z is 0, then R₁₈ denotes R₂₂.

The alkyl radicals R₁₅ may be linear or branched, and more particularly linear.

Preferably, R₁₅ denotes a methyl, ethyl, hydroxyethyl or dihydroxypropyl radical, and more particularly a methyl or ethyl radical.

Advantageously, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

Advantageously, R₁₇, R₁₉ and R₂₁, which may be identical or different, are chosen from linear or branched, saturated or unsaturated C₁-C₂, hydrocarbon-based radicals, and more particularly from linear or branched, saturated or unsaturated C₁₁-C₂, alkyl and alkenyl radicals.

Preferably, x and z, which may be identical or different, are 0 or 1.

Advantageously, y is equal to 1.

Preferably, r, n and p, which may be identical or different, are equal to 2 or 3 and even more particularly equal to 2.

The anion X⁻ is preferably a halide (chloride, bromide or iodide) or a C₁-C₄ alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate.

Use is made more particularly in the composition according to the invention of the ammonium salts of formula (IV) in which:
R₁₅ denotes a methyl or ethyl radical,
x and y are equal to 1;
z is equal to 0 or 1;
r, n and p are equal to 2;
R₁₆ is chosen from:
a radical 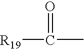
methyl, ethyl or C₁₄-C₂₂ hydrocarbon-based radicals,
a hydrogen atom;
R₁₈ is chosen from:
a radical 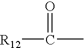
a hydrogen atom;
R₁₇, R₁₉ and R₂₁, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are advantageously linear.

Examples of compounds of formula (VIII) that may be mentioned include the salts (especially chloride or methyl sulfate) of diacyloxyethyl-dimethylammonium, of diacyloxyethyl-hydroxyethyl-methylammonium, of monoacyloxyethyl-dihydroxyethyl-methyl-ammonium, of triacyloxyethyl-methylammonium, of monoacyloxy-ethyl-hydroxyethyl-dimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

Mention may also be made of the salts of esters comprising a quaternized nitrogen in their structure, for instance N,N-dipalmitoyloxyethyl or N,N-dimethylammonium chloride, N,N-dihydrogenopalmitoyloxyethyl or N-hydroxyethyl-N-methylammonium methosulfate and distearyloxyethyldimethylammonium chloride.

The cationic surfactants according to the invention may preferably comprise a mixture of quaternary ammonium mono-, di- and triester salts with a weight majority of diester salts.

Examples of mixtures of ammonium salts that may be used include the mixture containing 15% to 30% by weight of acyloxyethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in U.S. Pat. No. 4,874,554 and U.S. Pat. No. 4,137,180.

Among the quaternary ammonium salts mentioned above that are preferably used are those corresponding to formula (V). Mention may be made firstly of tetraalkylammonium chlorides, for instance dialkyldimethylammonium or alkyltrimethylammonium chlorides in which the alkyl radical contains from about 12 to 22 carbon atoms, in particular behenyltrimethylammonium, distearyldimethylammonium, cetyltrimethylammonium or benzyldimethylstearylammonium chloride, or alternatively, secondly of palmitylamidopropyltrimethylammonium chloride or stearamidopropyldimethyl(myristyl acetate)ammonium chloride sold under the name Ceraphyl® 70 by the company Van Dyk.

The cationic surfactants that are particularly preferred in the composition of the invention are chosen from quaternary ammonium salts, and in particular from behenyltrimethylammonium chloride, cetyltrimethylammoniumchloride, quaternium-83, behenylamido-propyl-2,3-dihydroxypropyldimethylammonium chloride and palmityl-amidopropyltrimethylammonium chloride.

The cationic conditioning agent(s) may also be chosen from cationic silicones, and especially cationic amino silicones.

According to the invention, the term “aminosilicone” denotes any silicone containing at least one primary, secondary or tertiary amine or a quaternary ammonium group. Mention may thus be made of:
(a) the polysiloxanes referred to in the CTFA dictionary as “amodimethicone” and corresponding to the formula: 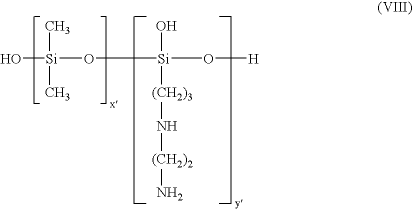
in which x′ and y′ are integers dependent on the molecular weight, generally such that the said number-average molecular weight is between 5000 and 500 000 approximately;
(b) aminosilicones corresponding to the formula:
   R′ₐG₃₋ₐ—Si(OSiG₂)ₙ—(OSiG_{b}R′_{2-b})ₘ—O—SiG₃₋ₐ—R′ₐ (IX)
in which:

G is a hydrogen atom or a phenyl, OH or C₁-C₈ alkyl, for example methyl, group,
a denotes the number 0 or an integer from 1 to 3, in particular 0,
b denotes 0 or 1, and in particular 1,
m and n are numbers such that the sum (n+m) can range especially from 1 to 2000 and in particular from 50 to 150, n possibly denoting a number from 0 to 1999 and in particular from 49 to 149 and m possibly denoting a number from 1 to 2000, and in particular from 1 to 10;
R′ is a monovalent radical of formula —C_{q}H_{2q}L in which q is a number from 2 to 8 and L is an optionally quaternized amine group chosen from the groups:
—NR″—CH₂—CH₂—N′(R″)₂
—N(R″)₂
—N^{⊕}(R″)₃A⁻
—NH^{⊕}(R″)₂A⁻
—NH₂^{⊕}(R″)A⁻
—N(R″)—CH₂—CH₂—N^{⊕}R″H₂A⁻,
in which R″ can denote hydrogen, phenyl, benzyl or a saturated monovalent hydrocarbon-based radical, for example an alkyl radical containing from 1 to 20 carbon atoms, and A⁻ represents a halide ion such as, for example, fluoride, chloride, bromide or iodide.

A product corresponding to this definition is the silicone known as “trimethylsilyl amodimethicone”, corresponding to the formula: 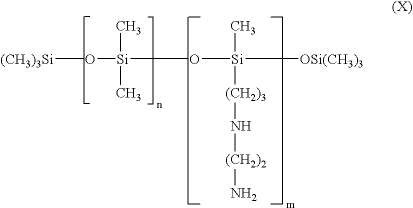
in which n and m have the meanings given above (cf. formula IX).

Such polymers are described, for example, in patent application EP-A-9523 8.

(c) aminosilicones corresponding to the formula: 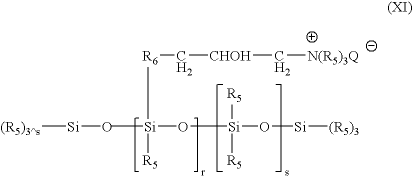
in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q⁻ is an anion such as a halide ion, in particular chloride, or an organic acid salt (acetate, etc.);
r represents an average statistical value from 2 to 20 and in particular from 2 to 8;
s represents an average statistical value from 20 to 200 and in particular from 20 to 50.

Such aminosilicones are described more particularly in patent U.S. Pat. No. 4,185,087.
d) quaternary ammonium silicones of formula: 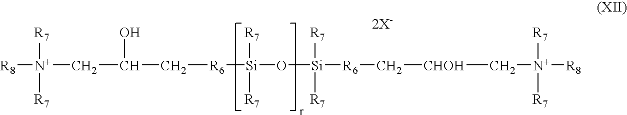
in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide ion, in particular chloride, or an organic acid salt (acetate, etc.);
r represents an average statistical value from 2 to 200 and in particular from 5 to 100.

These silicones are described, for example, in application EP-A-0 530 974.
e) aminosilicones of formula (XIII): 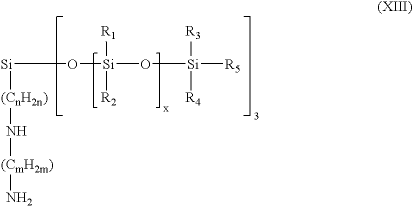
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,
and in which x is chosen such that the amine number is between 0.01 and 1 meq/g.

The silicones that are particularly preferred in accordance with the invention are:
polysiloxanes containing amine groups, such as amodimethicones or trimethylsilyl amodimethicones (CTFA 4th edition 1997).

The cationic conditioning agent(s) may also be chosen from cationic proteins.

The animal or plant cationic proteins or protein hydrolysates are in particular chemically modified polypeptides bearing at the end of the chain, or grafted thereto, quaternary ammonium groups, for instance trialkylammonium groups, the alkyl group being of C₁-C₃₀, or dialkylarylammonium groups, the alkyl group being of C₁-C₃₀ and the aryl group preferably being a phenyl or benzyl group. Their molecular mass can range, for example, from 1500 to 10 000 and in particular from 2000 to 5000 approximately. Among these compounds, mention may be made in particular of:
collagen hydrolysates bearing triethylammonium groups, such as the products sold under the name Quat-Pro E by the company Maybrook and referred to in the CTFA dictionary as “Triethonium Hydrolyzed Collagen Ethosulfate”;
collagen hydrolysates bearing trimethylammonium and trimethylstearylammonium chloride groups, sold under the name Quat-Pro S by the company Maybrook and referred to in the CTFA dictionary as “Steartrimonium Hydrolyzed Collagen”;
animal protein hydrolysates bearing trimethylbenzyl-ammonium groups such as the products sold under the name Crotein BTA by the company Croda and referred to in the CTFA dictionary as “Benzyltrimonium hydrolyzed animal protein”;
protein hydrolysates bearing, on the polypeptide chain, quaternary ammonium groups containing at least one alkyl radical having from 1 to 18 carbon atoms.

Among these protein hydrolysates, mention may be made, inter alia, of:
Croquat L in which the quaternary ammonium groups contain a C₁₋₂ alkyl group;
Croquat M in which the quaternary ammonium groups contain C₁₀-C₁₈ alkyl groups;
Croquat S in which the quaternary ammonium groups contain a C₁₋₈ alkyl group;
Crotein Q in which the quaternary ammonium groups contain at least one alkyl group having from 1 to 18 carbon atoms.

These various products are sold by the company Croda.

Other quaternized proteins or hydrolysates are, for example, those corresponding to the formula: 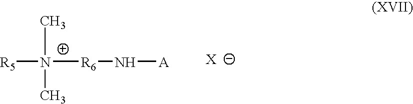
in which X⁻ is an anion of an organic or mineral acid, A denotes a protein residue derived from hydrolysates of collagen protein, R₅ denotes a lipophilic group containing up to 30 carbon atoms and R₆ represents an alkylene group having 1 to 6 carbon atoms. Mention may be made, for example, of the products sold by the company Inolex under the name Lexein QX 3000, referred to in the CTFA dictionary as “Cocotrimonium Collagen Hydrolysate”.

Mention may also be made of quaternized plant proteins such as wheat, corn or soybean proteins: as quaternized wheat proteins, mention may be made of those sold by the company Croda under the names Hydrotriticum WQ or QM, referred to in the CTFA dictionary as “Cocodimonium Hydrolysed Wheat Protein”, Hydrotriticum QL, referred to in the CTFA dictionary as “Lauridimonium Hydrolysed Wheat Protein” or Hydrotriticum QS, referred to in the CTFA dictionary as “Steardimonium Hydrolysed Wheat Protein”.

The conditioning agent(s) according to the invention may be used as a mixture with one or more solid or pasty, and preferably pulverulent, adjuvants. The adjuvants may be chosen from clays, salts, anionic, nonionic, amphoteric or zwitterionic surfactants, natural or synthetic thickeners, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol or fructose, zinc oxide, zirconium oxide, resin particles, for instance silicone or silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polydimethylsiloxane gums, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the conditioning agent(s) of the invention is (are) preferably in an amount ranging from 0.5% to 99% by weight, better still from 1% to 80% by weight and even more preferably from 2% to 60% by weight relative to the total weight of conditioning agent(s) and adjuvants.

The cosmetic composition for treating keratin materials obtained according to the process of the invention contains, besides the cationic conditioning agent(s) and the component(s) of the fluid, i.e. water and/or cosmetically acceptable solvent(s), optionally all or some of the adjuvant(s) present in the solid or pasty mixture.

The invention also relates to a composition that may be obtained via the process according to the invention, the composition preferably being free of preserving agents.

Using the preparation process of the invention, a cosmetic composition for treating keratin materials is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The cosmetic composition for treating keratin materials optionally resulting from the mixture(s) and/or addition(s) indicated above will be referred to hereinbelow as the final cosmetic treatment composition or final composition.

One particular mode of implementation of the invention consists in applying the composition obtained by means of a device not requiring any human intervention, and optionally equipped with a cooling means.

The amount of the cationic conditioning agent(s) present in the final cosmetic treatment composition obtained via the process of the present invention is generally between 0.001% and 50% by weight approximately, preferably between 0.005% and 30% and even more preferably between 0.01% and 20%, relative to the total weight of the final cosmetic treatment composition.

When the cosmetic composition obtained via the process of the present invention is mixed with a cosmetically acceptable medium, the medium generally consists of water or of a mixture of water and of at least one organic solvent to dissolve the compounds that would not be sufficiently soluble in water.

Examples of organic solvents that may be mentioned include C₁-C₄ lower alcohols, such as ethanol and isopropanol; polyols and polyol ethers, for instance 2-butoxyethanol, propylene glycol, propylene glycol monomethyl ether and diethylene glycol monomethyl ether and monoethyl ether, and also aromatic alcohols, for instance benzyl alcohol or phenoxyethanol, and mixtures thereof.

The solvents are preferably present in proportions preferably of between 1% and 40% by weight and even more preferably between 5% and 30% by weight relative to the total weight of the final composition.

At least one additive conventionally used in cosmetics may also be added to the cosmetic treatment compositions obtained according to the process of the present invention. Examples of such additives that may be mentioned include anionic, nonionic, amphoteric or zwitterionic surfactants, or mixtures thereof, anionic, nonionic, amphoteric or zwitterionic polymers, or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioning agents other than those described above, for instance non-cationic silicone oils, film-forming agents, ceramides, preserving agents and opacifiers, and also oils, waxes, gums and coloured or nacreous pigments.

The above additives are generally present in an amount for each of them of between 0.01% and 20% by weight relative to the weight of the final composition.

Needless to say, a person skilled in the art will take care to select this or these optional compounds such that the advantageous properties intrinsically associated with the cosmetic composition in accordance with the invention are not, or are not substantially, adversely affected by the addition(s) envisaged.

The pH of the final composition is generally between 3 and 12 and preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as mono-, di- and triethanolamine and derivatives thereof, sodium hydroxide, potassium hydroxide and the compounds of formula (II) below: 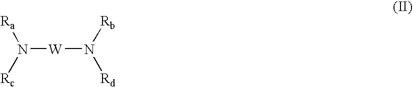
in which W is a propylene residue that is optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The final cosmetic treatment composition may be in various forms, such as in the form of liquids, creams or gels, or in any other form that is suitable for treating keratin materials, and especially keratin fibres, and the skin.

The final cosmetic treatment composition may be used, for example, as a shampoo, a rinse-out or leave-in conditioner, a deep-down care mask, a shower gel, or a lotion or cream for treating keratin materials.

The present invention also relates to a cosmetic process for treating keratin materials, comprising the preparation of a cosmetic treatment composition according to the process as defined above, and its application to the keratin materials, for example by means of an operator or by means of a device not requiring any human intervention. The application time may for example range between 15 seconds and one hour.

Before application, the cosmetic treatment composition obtained according to the process of the invention may be mixed with a cosmetically acceptable medium and/or with one or more additives conventionally used in cosmetics, as described above.

Another mode of implementation is preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The examples below are intended to illustrate the present invention.

### EXAMPLE 1

The ingredients below are mixed together in the proportions indicated as weight percentages relative to the total weight of solid mixture:

| | |
|---|---|
| stearyldimethylbenzylammonium chloride | 25% |
| non-pregelatinized corn distarch phosphate (1) | 50% |
| crystalline sorbitol | 25% |

(1) product sold under the name Corn Starch PO₄ by the company TR1 K

5 g of this mixture are placed in an espresso machine. The steam produced by the machine then passes through this solid mixture. The percolation is continued until 50 ml of a composition A are obtained. Composition A obtained may then be mixed with an aqueous composition B containing 1% hydroxyethylcellulose in a proportion of two parts of composition A per one part of composition B, to facilitate the application conditions.

A cosmetic treatment composition ready to be applied to the hair is thus obtained.

Smooth, shiny hair that feels soft and is easy to disentangle is obtained.

### EXAMPLE 2

The ingredients in powder form below are mixed together in the proportions indicated as weight percentages relative to the total weight of solid mixture:

| | |
|---|---|
| hydroxyethylcellulose (1) | 8% |
| lauryl monophosphate (2) | 82% |
| quaternized hydroxyethylcellulose (3) | 10% |

(1) product sold under the name Natrosol 250 HHR by the company Aqualon
(2) product sold under the name MAP 20 by the company KAO
(3) product sold under the name Celquat SC 240 C (28-6804) by the company National Starch

5 g of this mixture are placed in an espresso machine. The steam produced by the machine then passes through this solid mixture. The percolation is continued until 50 ml of a composition A are obtained. Composition A obtained may then be mixed with an aqueous composition B containing 1% hydroxyethylcellulose in a proportion of two parts of composition A per one part of composition B, to facilitate the application conditions.

A cosmetic treatment composition ready to be applied to the hair is thus obtained.

Smooth, shiny hair that feels soft and is easy to disentangle is obtained.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a cosmetic composition, comprising percolating a fluid at a pressure of at least 3 bar through at least one cationic conditioning agent for keratin materials, in solid or pasty form.

2. The process according to claim 1, characterized in that the cationic conditioning agent is chosen from cationic polymers, cationic surfactants, cationic silicones and cationic proteins.

3. The process according to claim 2, characterized in that the cationic polymer is chosen from:
(1) homopolymers or copolymers derived from acrylic or methacrylic esters or amides and comprising at least one of the units of the following formulae: 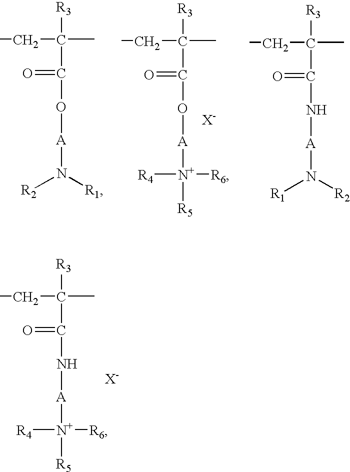
in which:
R₃, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical;
R₁ and R₂, which may be identical or different, represent hydrogen or an alkyl group containing from 1 to 6 carbon atoms;
X⁻ denotes an anion derived from a mineral or organic acid;
(2) cationic polysaccharides;
(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers;
(4) water-soluble polyamino amides prepared in particular by polycondensation of an acidic compound with a polyamine;
(5) polyaminoamide derivatives resulting from the condensation of polyalkylene polyamines with polycarboxylic acids followed by alkylation with difunctional agents;
(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms;
(7) cyclopolymers of alkyldiallylamine or of dialkyl-diallylammonium, in the form of homopolymers or copolymers;
(8) quaternary diammonium polymers containing repeating units corresponding to the formula: 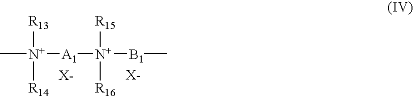
in which formula (IV):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals such as hydroxyethyl, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ—;
in which D denotes:
a) a glycol residue of formula: —O-Z-O—, where Z denotes a linear or branched hydrocarbon-based radical or a group corresponding to one of the following formulae:
—(CH₂—CH₂—O)ₙ—CH₂—CH₂—
—[CH₂—CH(CH₃)—O]_{y}—CH₂—CH(CH₃)—
where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical
—CH₂—CH₂-S—S—CH₂—CH₂—;
d) a ureylene group of formula: —NH—CO—NH—;
(9) polyquaternary ammonium polymers consisting of units of formula (IX): 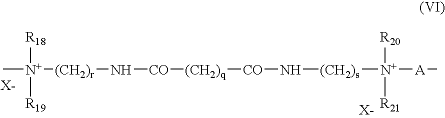
in which formula:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical,
where p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion such as a halide,
A denotes a dihalide radical or preferably represents —CH₂—CH₂-O—CH₂—CH₂—;
(10) quaternary polymers of vinylpyrrolidone and of vinylimidazole;
(11) polyamines;
(12) crosslinked methacryloyloxy(C₁-C₄)alkyltri(C₁-C₄)alkyl-ammonium salt polymers;
(13) polyalkyleneimines, polymers containing vinylpyridine or vinylpyridinium units, condensates of polyamines and of epichlorohydrin, quaternary polyureylenes, cationic polyurethanes, and chitin derivatives.

4. The process according to claim 2, characterized in that the cationic polymer is chosen from quaternary cellulose ether derivatives, cationic cyclopolymers, quaternary polymers of vinylpyrrolidone and of vinylimidazole, and crosslinked homopolymers or copolymers of methacryloyloxy(C₁-C₄)alkyltri(C₁-C₄)alkylammonium salts, and mixtures thereof.

5. The process according to claim 2, characterized in that the cationic surfactant is chosen from quaternary ammonium salts, quaternary ammonium salts of imidazoline, diquaternary ammonium salts and quaternary ammonium salts containing at least one ester function.

6. The process according to claim 5, characterized in that the quaternary ammonium salts are chosen from behenyltrimethyl-ammonium chloride, cetyltrimethylammonium chloride, quaternium-82, behenylamidopropyl-2,3-dihydroxypropyl dimethylammonium chloride and palmitylamidopropyltrimethylammonium chloride.

7. The process according to claim 2, characterized in that the cationic silicones are chosen from:
(a) the polysiloxanes referred to in the CTFA dictionary as “amodimethicone” and corresponding to the formula: 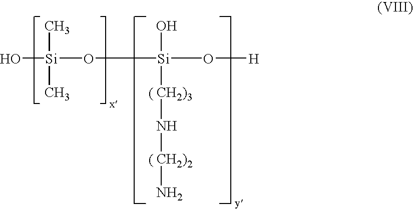
in which x′ and y′ are integers such that the said number-average molecular weight is between 5000 and 500 000 approximately;
(b) aminosilicones corresponding to the formula:
R′ₐG₃₋ₐ—Si(OSiG₂)ₙ—(OSiG_{b}R′_{2-b})ₘ—O—SiG₃₋ₐ—R′ₐ (IX)
in which:
G is a hydrogen atom or a phenyl, OH or C₁-C₈ alkyl,
a denotes the number 0 or an integer from 1 to 3,
b denotes 0 or 1,
m and n are numbers such that the sum (n+m) can range especially from 1 to 2000 and in particular from 50 to 150, n possibly denoting a number from 0 to 1999 and in particular from 49 to 149 and m possibly denoting a number from 1 to 2000, and in particular from 1 to 10;
R′ is a monovalent radical of formula —C_{q}H_{2q}L in which q is a number from 2 to 8 and L is an optionally quaternized amine group chosen from the groups:
—NR″—CH₂—CH₂—N′(R″)₂
—N(R″)₂
—N^{⊕}(R″)₃A⁻
—N^{⊕}(R″)₃A⁻
—N^{⊕}(R″)₃A⁻
—N(R″)—CH₂—CH₂—N^{⊕}R″H₂A⁻,
in which R″ can denote hydrogen, phenyl, benzyl or a saturated monovalent hydrocarbon-based radical, and A⁻ represents a halide ion;
(c) aminosilicones corresponding to the formula: 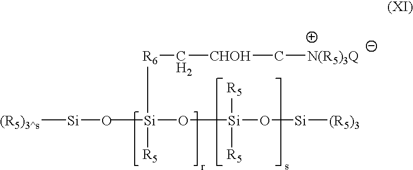
in which:
R₅ represents a monovalent hydrocarbon-based radical containing from 1 to 18 carbon atoms;
R₆ represents a divalent hydrocarbon-based radical;
Q⁻ is an anion;
r represents an average statistical value from 2 to 20;
s represents an average statistical value from 20 to 200;
d) quaternary ammonium silicones of formula: 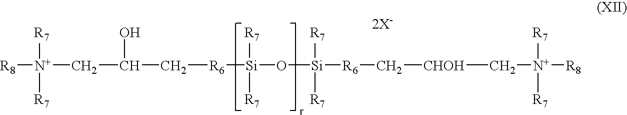
in which:
R₇, which may be identical or different, represent a monovalent hydrocarbon-based radical containing from 1 to 18 carbon atoms;
R₆ represents a divalent hydrocarbon-based radical;
R₈, which may be identical or different, represent a hydrogen atom, a monovalent hydrocarbon-based radical containing from 1 to 18 carbon atoms;
X⁻ is an anion;
r represents an average statistical value from 2 to 200;
e) aminosilicones of formula (XIII): 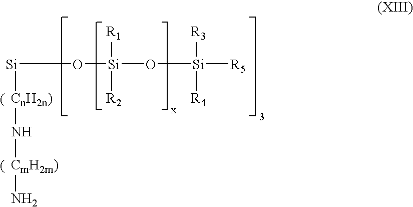
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,
and in which x is chosen such that the amine number is between 0.01 and 1 meq/g.

8. The process according to claim 2, characterized in that the cationic proteins are chosen from chemically modified polypeptides bearing at the end of the chain, or grafted thereto, quaternary ammonium groups chosen from trialkylammonium groups, the alkyl group being of C₁-C₃₀, and dialkylarylammonium groups, the alkyl group being of C₁-C₃₀ and the aryl group being a phenyl or benzyl group.

9. The process according to claim 8, characterized in that the cationic proteins are chosen from the collagen hydrolysates bearing triethylammonium groups, collagen hydrolysates bearing triethylammonium chloride and trimethylstearylammonium chloride groups, animal protein hydrolysates bearing trimethylbenzylammonium groups, and protein hydrolysates bearing on the polypeptide chain quaternary ammonium groups bearing at least one alkyl radical containing from 1 to 18 carbon atoms.

10. The process according to claim 1, characterized in that the cationic conditioning agent in solid or pasty form is mixed with an adjuvant.

11. The process according to claim 8, characterized in that the adjuvant is chosen from clays, salts, anionic, nonionic or zwitterionic surfactants, natural or synthetic thickeners, starch, optionally modified, glass beads, silica, Nylon, alumina, titanium dioxide, zeolites, polymethyl methacrylate (PMMA), chitosan, maltodextrin, cyclodextrin, mono- or disaccharides, zinc oxide, zirconium oxide, resin particles, for instance silicone or silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polydimethylsiloxane gums, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles.

12. The process according to claim 10, characterized in that the cationic conditioning agent(s) is (are) present in an amount ranging from 0.5% to 99% by weight, preferably from 1% to 80% and even more preferably from 2% to 60% by weight relative to the total weight of cationic conditioning agent(s) and adjuvant.

13. The process according to claim 1, characterized in that the percolation step is performed with a fluid at a pressure of at least 10 bar.

14. The process according to claim 1, characterized in that the fluid is steam, optionally accompanied by liquid water.

15. The process according to claim 1, characterized in that the fluid consists of one or more cosmetically acceptable liquid and/or gaseous organic solvents.

16. A cosmetic composition for treating keratin materials, obtained via the process according to claim 1.

17. The composition according to claim 16, wherein said composition is free of preserving agent.

18. A process for treating keratin material, comprising applying the composition of claim 16 to keratin material.

19. The process according to claim 18, wherein said composition is applied with a device not requiring any human intervention.

20. The process according to claim 18, wherein, before application, the composition is mixed with a cosmetically acceptable medium and/or with one or more additives used in cosmetics.

21. A process for treating keratin material, wherein at least two cosmetic compositions are prepared according to the process according to claim 1 are mixed together and the mixture is applied to the keratin material.

22. A device, comprising a closed housing delimited by at least one wall that is at least partially permeable to a fluid at a pressure of at least 3 bar, the device comprising therein at least one cationic conditioning agent in solid or pasty form.

23. The device according to claim 22, wherein the housing is delimited by two sealed sheets.

24. The device according to claim 22, wherein the housing is delimited by a tray closed with a lid.
